(19) United States
(12) Patent Application Publication
(10) Pub. No.: US 2022/0347346 A1
(43) Pub. Date: Nov. 3, 2022

(54) MESENCHYMAL STEM CELL SHEET AND USE THEREOF

(71) Applicants: BOE REGENERATIVE MEDICINE TECHNOLOGY CO., LTD., Beijing (CN); BOE TECHNOLOGY GROUP CO., LTD., Beijing (CN)

(72) Inventors: Dehua CHANG, Beijing (CN); Jianlin MA, Beijing (CN); Shuang GAO, Beijing (CN); Juan WANG, Beijing (CN); Jing WANG, Beijing (CN); Xin JIN, Beijing (CN); Shuai LIU, Beijing (CN); Donghua LIU, Beijing (CN); Yufei ZHAO, Beijing (CN); Yang LIU, Beijing (CN); Yuqin TAN, Beijing (CN)

(21) Appl. No.: 17/430,086

(22) PCT Filed: Jan. 22, 2021
(86) PCT No.: PCT/CN2021/073295

§ 371 Date: Aug. 11, 2021

(30) Foreign Application Priority Data
Jan. 22, 2020 (CN) PCT/CN2020/073765

(51) Int. Cl. A61L 27/38; A61L 27/50; A61P 9/00; C12N 5/0775

(52) U.S. Cl. CPC A61L 27/38; A61L 27/50; A61P 9/00; C12N 5/0665

(57) ABSTRACT

Provided is a method for treating diseases related to cardiac tissue damage or cardiac insufficiency in a subject. The method includes the step of locally applying a mesenchymal stem cell sheet such as an umbilical cord mesenchymal stem cell sheet to the heart of the subject. Also provided are related use and compositions of the mesenchymal stem cell sheet.

This application claims the priority of PCT application with the application number PCT/CN2020/073765, titled “MESENCHYMAL STEM CELL SHEETS AND USE THEREOF” submitted on Jan. 22, 2020. The entire content of the above PCT application disclosure is incorporated by reference as a part of the present application.

### INVENTION FIELD

The present disclosure relates to the field of tissue engineering and regenerative medicine, and in particular to use of a mesenchymal stem cell sheet (such as an umbilical cord mesenchymal stem cell) in the treatment of a disease related to cardiac tissue damage or cardiac insufficiency in a subject.

### TECHNICAL BACKGROUND

The results of the cardiovascular disease survey show that the current prevalence of heart failure in adults in our country is 0.9%. According to the Blue Book Report of Heart Failure in China in 2019, it is estimated that the number of patients with heart failure in our country is as high as 6.5-8.75 million, and the number is increasing by 200,000 per year. Cardiovascular risk factors such as aging of the population, hypertension and hyperlipidemia are the main causes of heart failure. The prevalence of heart failure in people over 70 years old is >10%. The 5-year mortality rate in patients with heart failure is 50%, and the 1-year mortality rate in patients with severe heart failure can reach 50%.

Almost all cardiovascular diseases will eventually lead to heart failure. Myocardial infarction, cardiomyopathy and the like cause myocardial damage due to ischemia, hemodynamic overload, inflammation, etc., which in turn causes changes in myocardial structure and function, and finally leads to poor ventricular pumping and filling, resulting in heart failure. Although chemotherapy, interventional techniques, and surgical treatment have significantly reduced the mortality rate of acute myocardial infarction, clinical observations have found that even in patients with acute myocardial infarction who successfully receive early revascularization treatment, more than 30% of the patients will eventually develop ischemic heart failure. Currently, there are only two effective treatments for end-stage heart failure: heart transplantation and implantation of an artificial heart (ventricular assist device). Because of the severe shortage of donors, heart transplantation cannot meet the needs of many patients waiting for heart transplantation. An artificial heart is only used as a bridge of short-term transition for patients with heart failure. Moreover, it is expensive and prone to result in complications such as infection and bleeding after surgery. Therefore, there is no effective way to completely solve the condition of patients with severe heart failure in the international medical community.

In recent years, the development of stem cells and regenerative medicine has brought hope to patients with severe heart failure. Previous animal experiments and clinical studies have found that stem cells of various sources and types can effectively treat acute and chronic myocardial infarction, ischemic heart failure, etc. Through the paracrine action of stem cells, the contraction and relaxation of the heart are improved, myocardial fibrosis is prevented, and the prognosis is improved. However, stem cell therapy mentioned above still has the following problems in the clinical application: there are ethical, legal, and immune rejection issues on embryonic stem cells; iPS cells are potentially oncogenic and requires HLA matching; skeletal myoblasts can cause ventricular arrhythmia; bone marrow or peripheral blood mononuclear cells have complex components, with immune cells as the dominance, and few stem cells with multidirectional differentiation potential contained therein; bone marrow mesenchymal stem cells (MSCs) are limited in source and their cell proliferation capacity and stemness decreases significantly with age.

Mesenchymal stem cells are widely present in human tissues. They have the ability to expand in vitro, multiple differentiation potentials and immunomodulatory effects, which have been used in self-tissue repair and the treatment of immune-related diseases. Currently, in the basic research and clinical application of mesenchymal stem cells on the self-tissue repair, direct injection of cells or transplantation after the combination of cells and materials of scaffold for tissue engineering are employed in most methods, but both of them have certain limitations. Direct injection of cells will cause loss of a large number of cells, thus leading to low efficiency of the treatment, and the function of stem cells in tissue repair is limited. The transplantation after combination of cells and scaffold for tissue engineering solves the problem of cell loss though, the scaffold materials may cause inflammation in the body to various extents, and the degradation process and products of the scaffold may cause local lesions in tissue.

Therefore, there are currently no ideal stem cell products that can meet the clinical needs of patients with severe heart failure.

A cell sheet is a new method of cell therapy in recent years, which can form two-dimensional and three-dimensional structures through the connections between cells alone. Compared with traditional single-cell suspensions or methods that combine cells with tissue engineering scaffold, a cell sheet can better achieve local fixation and reduce cell loss, thereby improving cell utilization and avoiding heterologous substances that may trigger a large immune response introduced by the materials of the scaffold.

### SUMMARY

The inventors prepared mesenchymal stem cell sheets and evaluated them in the constructed animal model of heart failure. The results showed that mesenchymal stem cell sheets of the present invention have a good therapeutic effect on heart failure, improve the movement and ejection ability of the heart, and reduce cardiac remodeling and fibrosis.

Accordingly, in the first aspect, the present disclosure relates to a method for treating a disease related to cardiac tissue damage or cardiac insufficiency in a subject, wherein the method comprises steps of topically applying mesenchymal stem cell sheets to the heart of the subject.

In some embodiments, the disease can be selected from ischemic heart disease, rheumatic heart disease, congenital heart disease, cardiomyopathy, coronary heart disease and valvular heart disease.

In some embodiments, the cardiomyopathy is dilated cardiomyopathy.

In some embodiments, the disease can be ischemic heart failure, such as acute ischemic heart failure, chronic ischemic heart failure or end-stage ischemic heart failure.

In some embodiments, the disease is chronic heart failure caused by ischemic heart disease and dilated cardiomyopathy. For example, one is heart failure caused by myocardial ischemia, including chronic ischemic heart failure and end-stage ischemic heart failure, and the other is heart failure caused by dilated cardiomyopathy.

In some embodiments, the mesenchymal stem cell sheet is attached to anterior or lateral wall of left ventricle of heart.

In some embodiments, the mesenchymal stem cell sheet is attached to damaged or defective sites of heart or adjacent sites thereof for the treatment of the disease related to cardiac tissue damage or cardiac insufficiency. In other embodiments, the mesenchymal stem cell sheet is implanted to damaged or defective sites of heart, or adjacent sites thereof. Compared with traditional single-cell suspensions or methods that combine cells with materials of scaffold for tissue engineering, cell sheets can better achieve local fixation and reduce cell loss, thereby improving cell utilization rate and at the same time avoiding heterologous substances that may trigger a large immune response introduced by the materials of the scaffold.

In some embodiments, the cell ratio of mesenchymal stem cells in the mesenchymal stem cell sheet can be at least 90%. In some embodiments, the cell ratio of mesenchymal stem cells in the sheet is at least 95%, such as at least 96%, at least 97%, at least 98%, or at least 99%, e.g., as identified by detecting cell surface markers through flow cytometry, three-direction differentiation assay, or detecting gene expression in cells through PCR method.

In some embodiments of the methods above, the mesenchymal stem cells can be derived from a tissue selected from amniotic fluid, amniotic membrane, chorion, chorionic villus, decidua, placenta, umbilical cord blood, Wharton's jelly, umbilical cord, adult bone marrow, dental pulp, adult peripheral blood and adult adipose tissue.

In some embodiments of the methods above, the mesenchymal stem cells can be selected from umbilical cord mesenchymal stem cells, placental mesenchymal stem cells, adipose mesenchymal stem cells, dental pulp mesenchymal stem cells and bone marrow mesenchymal stem cells. For example, the mesenchymal stem cells can be umbilical cord mesenchymal stem cells.

In some embodiments, the mesenchymal stem cell sheet can be prepared by using mesenchymal stem cells with passage number of P0-P20. For example, cell sheets can be prepared by using mesenchymal stem cells with passage number of P2-P15, P2-P8, or P2-P10.

In some embodiments, the thickness of the mesenchymal stem cell sheet can be about 10-300 μm, such as the thickness of 30-300 μm, 50-300 μm, 100-300 μm, 80-300 μm, 100-300 μm, 120-300 μm, 150-300 μm or 200-300 μm. In some embodiments, the thickness of the mesenchymal stem cell sheet can be about 15-250 μm.

In addition, the mesenchymal stem cell sheet in the present disclosure can have different layers of cells. In some embodiments, the mesenchymal stem cell sheet can have 1-15 layers of cells, such as 2-15 layers, 3-15 layers, 5-15 layers of cells, 8-15 layers, or 10-15 layers of cells.

In some embodiments, the cell density of the mesenchymal stem cell sheet can be about 1×10⁵ to 5×10⁷/cm², such as about 1×10⁵ to 1×10⁷/cm², 8×10⁵ to 5×10⁷/cm² and 3×10⁵ to 5×10⁶/cm².

In the method of the present disclosure, the size and shape of a mesenchymal stem cell sheet used can be determined according to actual needs, e.g., according to the size and shape of damaged or defective sites of heart in a subject. In some embodiments, a mesenchymal stem cell sheet in a round shape or a shape that facilitates attachment or implantation can be used. In some embodiments, a round cell sheet with a diameter of 15-55 mm and a thickness of 10-300 μm can be used, attached to anterior or lateral wall of left ventricle of heart for the treatment of the disease related to cardiac tissue damage or cardiac insufficiency.

In some embodiments, the mesenchymal stem cells in the sheet are connected to each other by extracellular matrix secreted by the cells, wherein the extracellular matrix is rich in fibronectin and integrin-β1.

In some embodiments, the mesenchymal stem cells in the sheet are capable of secreting a variety of cytokines, such as angiogenic factors and immunomodulatory factors, such as one or more of hepatocyte growth factor (HGF), fibroblast growth factor (FGF), epidermal growth factor (EGF), transforming growth factor-β (TGF-β), prostaglandin E2 (PGE2), interleukin-6 (IL-6), interleukin-10 (IL-10), interleukin-8 (IL-8), vascular endothelial growth factor (VEGF), and platelet-derived growth factor (PDGF).

In some embodiments, 2×10⁷ to 8×10⁷ mesenchymal stem cells are comprised in the sheet.

In some embodiments, 1-4 of the mesenchymal stem cell sheets are topically applied to the heart of the subject.

In some embodiments, the mesenchymal stem cell sheet is topically applied to the heart of the subject in combination with coronary artery bypass graft performed on the subject, e.g., the mesenchymal stem cell sheet is topically applied to the heart of the subject after the coronary artery bypass graft is performed on the subject.

In some embodiments, the method of applying transplantation mesenchymal stem cell sheets in present invention comprises (1) thoracotomy is performed to open the pericardium, and 1-4 pieces of UCMSCS are applied to anterior and lateral walls of left ventricle of heart; (2) UCMSCS are applied to anterior and lateral walls of left ventricle of heart after coronary artery bypass graft; (3) UCMSCS are applied to anterior and lateral walls of left ventricle of heart through minimally invasive surgery.

In some embodiments, mesenchymal stem cells in the mesenchymal stem cell sheet are autologous or allogeneic to the subject.

In some embodiments of the method above, the mesenchymal stem cell sheet can be prepared by a method comprising the following steps:
a. culturing the mesenchymal stem cells in a thermo-sensitive petri dish with pre-coated adhesion matrix or serum;
b. detaching the mesenchymal stem cells from the thermo-sensitive petri dish by decreasing the temperature,

wherein the mesenchymal stem cell is connected to each other by extracellular matrix secreted by the cells, thereby obtaining said mesenchymal stem cell sheet.

In some embodiments, the mesenchymal stem cells used for preparation of a mesenchymal stem cell sheet can be made by a method comprising the following steps:
a. separating Wharton's jelly from umbilical cord;
b. shredding the Wharton's jelly into small tissue blocks, culturing the tissue blocks for enough period of time, and allowing mesenchymal stem cells to migrate from the tissue blocks;
c. removing the tissue blocks when the mesenchymal stem cells grow to about 50%-100% confluence, e.g., about 70%-100% confluence or about 80%-100% confluence, thereby obtaining umbilical cord mesenchymal stem cells; and optionally
d. culturing and passaging the umbilical cord mesenchymal stem cells.

As used herein, the term “thermo-sensitive petri dish” refers to a petri dish coated with a layer of thermo-sensitive polymer substance, of which molecular segments stretch distinctly at different temperatures, thereby exhibiting hydrophilicity or hydrophobicity, so that the hydrophilicity/hydrophobicity of the polymer substance can change with external temperature. When the surface of the thermo-sensitive petri dish is hydrophilic, the adhesion to the cells and the extracellular matrix secreted by the cells becomes poor, and the cells shed off in layers. In a specific application, when the temperature is lowered below the low critical solution temperature of the polymer substance, the surface of the thermo-sensitive petri dish exhibits hydrophilicity, so that the cells shed off in layers.

By utilizing a thermo-sensitive petri dish, lamellar mesenchymal stem cells are detached from the bottom of the thermo-sensitive petri dish without use of enzyme and analog digestion or physical peeling, then becoming a cell sheet with the complete connection of the extracellular matrix retained.

After culturing and harvesting the mesenchymal stem cells, the cell growth curve can be determined by MTT method, WST method, DNA content assay, ATP assay and the like to evaluate the growth activity of umbilical cord mesenchymal stem cells. In addition, the isolated and cultured mesenchymal stem cells can be identified by detecting cell surface markers through flow cytometry, three-direction differentiation assay, and detecting genes expression in cells through PCR method. In some embodiments, mesenchymal stem cells can be identified by detecting protein markers on the cell surface through flow cytometry.

In some embodiments of the methods above, the adhesion matrix used to coat the thermo-sensitive petri dish can include one or more of fetal bovine serum, autologous serum, collagen, gelatin, fibronectin, vitronectin, laminin, polyornithine, and polylysine.

In some embodiments of the methods above, the serum used to coat the thermo-sensitive petri dish is selected from fetal bovine serum (FBS) or human serum. In some embodiments, 100% serum is used as the coating solution. In other embodiments, basal medium (e.g., 1640, DMEM, α-MEM or DMEM/F12) containing at least 10% (v/v) serum is used as the coating solution.

In some embodiments of the methods above, the mesenchymal stem cells are detached from the thermo-sensitive petri dish by decreasing the temperature, thereby forming mesenchymal stem cell sheet. For example, in the case where the culture temperature is about 37° C., the mesenchymal stem cells are detached from the thermo-sensitive petri dish by lowering the temperature to 4-32° C. In some embodiments, 4° C. pre-chilled buffer (such as HBSS, PBS, or saline) is added to detach the mesenchymal stem cells from the thermo-sensitive petri dish.

In some embodiments, the mesenchymal stem cell sheet has an upper surface not contacted with the petri dish and a basal surface contacted with the petri dish during the preparation process, wherein the basal surface is rough. Due to the structural characteristics of the mesenchymal stem cell sheet, its the basal surface can provide greater friction, which is beneficial to the cell sheet to be better attached to the application site.

Therefore, in some embodiments, the basal surface of the mesenchymal stem cell sheet can be attached to damaged or defective sites of heart, or adjacent sites thereof.

In some embodiments, the basal surface of the mesenchymal stem cell sheet can be attached to anterior or lateral wall of left ventricle of heart.

In the second aspect, the present disclosure relates to use of the mesenchymal stem cell sheet in the treatment of a disease related to cardiac tissue damage or cardiac insufficiency in a subject, wherein the mesenchymal stem cell sheet is topically applied to the heart of the subject.

In the third aspect, the present disclosure relates to use of the mesenchymal stem cell sheet in the preparation of a composition for the treatment of a disease related to cardiac tissue damage or cardiac insufficiency in a subject, wherein the mesenchymal stem cell sheet is topically applied to the heart of the subject.

In some embodiments of the uses in the second and third aspects of the present disclosure, the disease can be selected from ischemic heart disease, rheumatic heart disease, congenital heart disease, cardiomyopathy, coronary heart disease and valvular heart disease.

In some embodiments, the cardiomyopathy is dilated cardiomyopathy.

In some embodiments of the uses in the second and third aspects of the present disclosure, the disease can be ischemic heart failure, such as acute ischemic heart failure, chronic ischemic heart failure or end-stage ischemic heart failure.

In some embodiments of the uses above, the mesenchymal stem cell sheet is attached to damaged or defective sites of heart, or adjacent sites thereof. In other embodiments, the mesenchymal stem cell sheet is implanted to damaged or defective sites of heart, or adjacent sites thereof. In some embodiments, the mesenchymal stem cell sheet is attached to anterior or lateral wall of left ventricle of heart.

In some embodiments of the uses above, the cell ratio of mesenchymal stem cells in the mesenchymal stem cell sheet can be at least 90%. In some embodiments, the cell ratio of the mesenchymal stem cells in the mesenchymal stem cell sheet is at least 95%, such as at least 96%, at least 97%, at least 98%, or at least 99%, e.g., as identified by detecting cell surface markers through flow cytometry, three-direction differentiation assay, or detecting gene expression in cells through PCR method.

In some embodiments of the uses above, the mesenchymal stem cells can be derived from a tissue selected from amniotic fluid, amniotic membrane, chorion, chorionic villus, decidua, placenta, umbilical cord blood, Wharton's jelly, umbilical cord, adult bone marrow, dental pulp, adult peripheral blood and adult adipose tissue.

In some embodiments of the uses above, the mesenchymal stem cells can be selected from the umbilical cord mesenchymal stem cells, placental mesenchymal stem cells, adipose mesenchymal stem cells, dental pulp mesenchymal stem cells and bone marrow mesenchymal stem cells. For example, the mesenchymal stem cells can be umbilical cord mesenchymal stem cells.

In some embodiments of the uses above, the mesenchymal stem cell sheet can be prepared by using mesenchymal stem cells with passage number of P0-P20. For example, the cell sheet can be prepared by using mesenchymal stem cells with passage number of P2-P15, P2-P8, or P2-P10.

In some embodiments of the uses above, the thickness of the mesenchymal stem cell sheet can be about 10-300 μm. In some embodiments, the thickness of the mesenchymal stem cell sheet can be about 20-300 μm, such as the thickness of 30-300 μm, 50-300 μm, or 100-300 μm. In addition, the mesenchymal stem cell sheet disclosed in present invention can have different layers of cells. In some embodiments, the mesenchymal stem cell sheet can have 1-15 layers of cells, such as 2-15 layers, 3-15 layers, or 5-15 layers of cells.

In some embodiments of the uses above, the cell density of the mesenchymal stem cell sheet can be about 1×10⁵ to 5×10⁷/cm², such as 8×10⁵ to 5×10⁷/cm² and 3×10⁵ to 5×10⁶/cm².

In some embodiments, mesenchymal stem cells in the sheet are connected to each other by extracellular matrix secreted by the cells, wherein the extracellular matrix is rich in fibronectin and integrin-01.

In some embodiments, the mesenchymal stem cells in the sheet are capable of secreting a variety of cytokines, such as angiogenic factors and immunomodulatory factors, such as one or more of HGF, EGF, FGF, TGF-0, PGE2, IL-6, IL-8, IL-10, PDGF and VEGF.

In some embodiments, 2×10⁷ to 8×10⁷ mesenchymal stem cells are comprised in the sheet.

In some embodiments, 1-4 of the mesenchymal stem cell sheets are topically applied to the heart of the subject.

In some embodiments, the mesenchymal stem cell sheet is topically applied to the heart of the subject in combination with coronary artery bypass graft performed on the subject, e.g., the mesenchymal stem cell sheet is topically applied to the heart of the subject after the coronary artery bypass graft is performed on the subject.

In some embodiments, mesenchymal stem cells in the mesenchymal stem cell sheet are autologous or allogeneic to the subject.

In some embodiments of the uses above, the mesenchymal stem cell sheet can be prepared by a method comprising the following steps:
a. culturing the mesenchymal stem cells in a thermo-sensitive petri dish pre-coated with adhesion matrix and/or serum;
b. detaching the mesenchymal stem cells from the thermo-sensitive petri dish by decreasing the temperature,

wherein the mesenchymal stem cells are connected to each other by extracellular matrix secreted by the cells, thereby obtaining said mesenchymal stem cell sheet.

In some embodiments of the uses above, the mesenchymal stem cells used for preparation of mesenchymal stem cell sheet can be made by a method comprising the following steps:
a. separating Wharton's jelly from umbilical cord;
b. shredding Wharton's jelly into small tissue blocks, culturing the tissue blocks for enough period of time, and allowing mesenchymal stem cells to migrate from the tissue blocks;
c. removing the tissue blocks when the mesenchymal stem cells grow to about 50%-100% confluence, e.g., about 70%-100% confluence or about 80%-100% confluence, thereby obtaining umbilical cord mesenchymal stem cells; and optionally
d. culturing and passaging the umbilical cord mesenchymal stem cells.

In some embodiments of the uses above, the adhesion matrix used to coat the thermo-sensitive petri dishes can include one or more of fetal bovine serum, autologous serum, collagen, gelatin, fibronectin, vitronectin, laminin, polyornithine, and polylysine.

In some embodiments of the uses above, the serum used to coat the thermo-sensitive petri dish is selected from fetal bovine serum (FBS) or human serum. In some embodiments, 100% serum is used as the coating solution. In other embodiments, basal medium (e.g., 1640, DMEM, α-MEM or DMEM/F12) containing at least 10% (v/v) serum is used as the coating solution.

In some embodiments of the uses above, the mesenchymal stem cells are detached from the thermo-sensitive petri dish by decreasing the temperature, thereby forming mesenchymal stem cell sheet. For example, in the case where the culture temperature is about 37° C., the mesenchymal stem cells are detached from the thermo-sensitive petri dish by lowering the temperature to 4-32° C. In some embodiments, 4° C. pre-chilled buffer is added to detach the mesenchymal stem cells from the thermo-sensitive petri dish.

In some embodiments of the uses above, the mesenchymal stem cell sheet has an upper surface not contacted with the petri dish and a basal surface contacted with the petri dish during the preparation process, wherein the basal surface is rough. In some embodiments, the basal surface of the mesenchymal stem cell sheet is attached to damaged or defective sites of heart, or adjacent sites thereof. In some embodiments, the basal surface of the mesenchymal stem cell sheet is attached to anterior or lateral wall of left ventricle of heart.

In some embodiments of the uses above, the mesenchymal stem cell sheet is a single-dose of mesenchymal stem cell sheet, wherein 2×10⁷ to 8×10⁷ mesenchymal stem cells are comprised in the sheet.

In some embodiments, the diameter of the single-dose of mesenchymal stem cell sheet is 15-55 mm and the thickness of the sheet is 10-300 μm.

In the fourth aspect, the present invention relates to a mesenchymal stem cell sheet for use in the treatment of a disease related to cardiac tissue damage or cardiac insufficiency in a subject.

In some embodiments, the cell density of the mesenchymal stem cell sheet is 8×10⁵ to 5×10⁷/cm².

In the fifth aspect, the present invention relates to a single-dose of mesenchymal stem cell sheet, wherein 2×10⁷ to 8×10⁷ mesenchymal stem cells are comprised in the sheet, wherein the mesenchymal stem cells are connected to each other by extracellular matrix secreted by the cells.

It should be noted that the “single-dose” of the present application refers to the dose that can exert a therapeutic effect every time using one mesenchymal stem cell sheet. According to the single-dose of mesenchymal stem cell sheet in the embodiments of the present invention, 1-4 sheets can be used at one time to be topically applied to the heart of the subject for the treatment of a disease related to cardiac tissue damage or cardiac insufficiency in a subject, e.g., can be attached to anterior or lateral wall of left ventricle of heart.

In some embodiments, the diameter of the single-dose of mesenchymal stem cell sheet is 15-55 mm and the thickness of the sheet is 10-300 μm.

In some embodiments, the cell density of the mesenchymal stem cell sheet is 8×10⁵ to 5×10⁷/cm².

In the sixth aspect, the present invention relates to a composition comprising mesenchymal stem cell sheets, wherein the composition is used for the treatment of a disease related to cardiac tissue damage or cardiac insufficiency in a subject.

In some embodiments of the fourth aspect, fifth aspect and sixth aspect in the present disclosure, the disease can be selected from ischemic heart disease, rheumatic heart disease, congenital heart disease, cardiomyopathy, coronary heart disease and valvular heart disease.

In some embodiments of the fourth aspect, fifth aspect and sixth aspect in the present disclosure, the disease can be ischemic heart failure, such as acute ischemic heart failure, chronic ischemic heart failure or end-stage ischemic heart failure.

In some embodiments of the fourth aspect, fifth aspect and sixth aspect in the present disclosure, the mesenchymal stem cells have one or more features described in the first, second, and third aspects of the present disclosure.

### BRIEF DESCRIPTION OF THE DRAWINGS

FIG. 1 shows a photograph of an umbilical cord mesenchymal stem cell sheet obtained according to the examples of the present invention.

FIG. 2 shows the results of detecting adipogenic and osteogenic differentiation functions of umbilical cord mesenchymal stem cells. FIG. 1A: The results of alizarin red staining FIG. 1B: The results of oil red O staining

FIG. 3 shows scanning electron microscope imaging photographs of the umbilical cord mesenchymal stem cell sheet. FIG. 3A: The surface (the upper surface) of the cell sheet. FIG. 3B: The basal surface of the cell sheet.

FIG. 4 shows immunofluorescence imaging photographs of the umbilical cord mesenchymal stem cell sheet. FIG. 4A: Fibronectin. FIG. 4B: Integrin-β1.

FIG. 5 shows results of detecting cytokine expressions in the culture supernatant of the umbilical cord mesenchymal stem cell sheet by using ELISA method.

FIG. 6 shows a photograph of a mesenchymal stem cell sheet transplanted to the mouse heart.

FIG. 7 shows the characterization of the constructed mouse disease model of heart failure. FIG. 7A: The photograph of the heart of the disease model mouse; FIG. 7B: Electrocardiogram results of the disease model mouse.

FIG. 8 shows photographs of the disease model mouse treated with the umbilical cord mesenchymal stem cell sheet. FIG. 8A shows an exemplary photograph of the mesenchymal stem cell sheet used; FIG. 8B shows a photograph of a cell sheet attached to the surface of the mouse heart.

FIG. 9 shows the echocardiogram results of mice at different time points. FIG. 9A: Before modeling; FIG. 9B: 1 week after modeling; FIG. 9C: 4 weeks after modeling. Left: animals in the control group; Right animals in the cell sheet transplantation group.

FIG. 10 shows the curves of the left ventricular ejection fraction of mice in the cell sheet treatment group and the control group over time.

FIG. 11 shows the curves of the left ventricular fractional shortening index of mice in the cell sheet treatment group and the control group over time.

FIG. 12 shows the curves of the left ventricular diameter of mice in the cell sheet treatment group and the control group over time.

FIG. 13 shows the curves of the left ventricular volume of mice in the cell sheet treatment group and the control group over time.

FIG. 14 shows the results of Masson staining of tissue sections of mouse heart after 28 days of cell sheet treatment. Left animals in the control group; right: animals in the cell sheet treatment group.

FIG. 15 shows a photograph of the anterior wall of left ventricle of heart of a miniature pig transplanted with 1-2 sheets of the obtained mesenchymal stem cell sheets.

FIG. 16 shows electrocardiograms before and after modeling of myocardial infarction in miniature pigs and after the treatment of cell sheet transplantation.

FIG. 17 shows statistical charts of changes in indicators such as EF, SV, LVFS, EDV, and ESV in the sheet transplantation group compared with the model control group.

FIG. 18 shows the pathological examination results for the myocardial infarction rate of animals in the model control group and the sheet transplantation group.

FIG. 19 shows the pathological examination results for the degree of myocardial fibrosis in the model control group and the sheet transplantation group.

### DETAILED DESCRIPTION

The technical problem to be solved by the present invention is to apply umbilical cord mesenchymal stem cell sheets of human origin to the treatment of patients with heart failure caused by ischemic heart disease, dilated cardiomyopathy, etc. The transplantation of cell sheets improves myocardial function, the quality of life, and reduces the mortality of patients with heart failure. At the same time, the method of sheet transplantation will overcome the defects that it can not be ensured that a sufficient number of stem cells reach the diseased site to fully exert their biological effects caused by the injection of mesenchymal stem cell suspension, and overcome the defects that stem cells+biological scaffolds induce inflammatory response due to the incomplete absorption of biological materials.

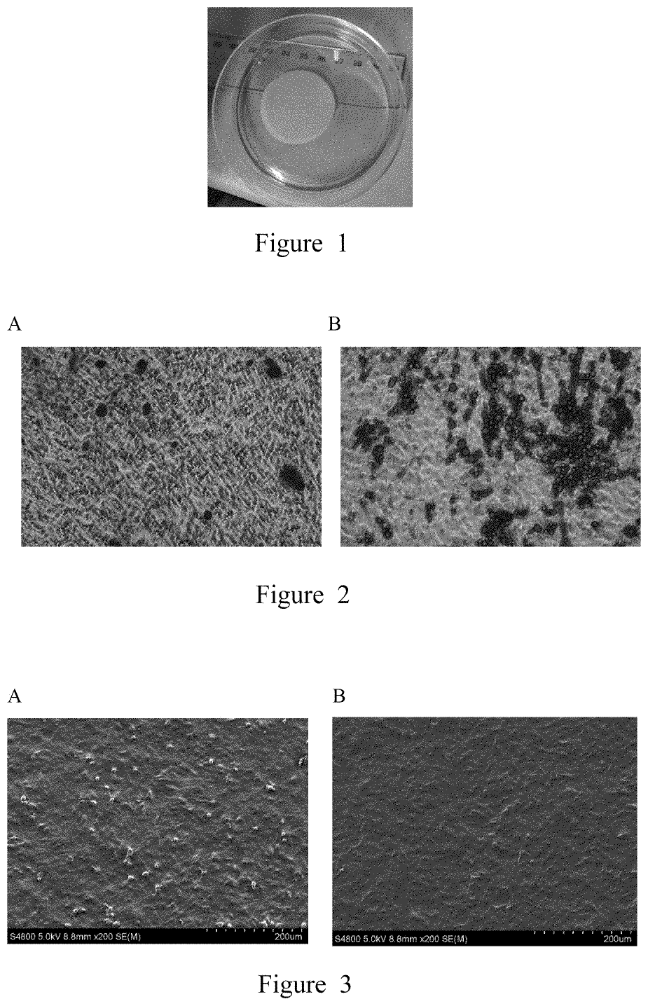

The “umbilical cord mesenchymal stem cell sheet” referred to in the present invention can be prepared according to the following method: take umbilical cord mesenchymal stem cells of P2-P8 passages to prepare a single-cell suspension, which is then added at a concentration of 3×10⁵-1.2×10⁶ cells/cm² to a thermo-sensitive petri dish that is pre-coated with matrix (such as fetal bovine serum, autologous serum, gelatin, fibronectin, vitronectin, laminin, polylysine). When the cells are cultured to supersaturated state, the thermo-sensitive petri dish is placed under the condition below 20° C., and the cells will spontaneously form a film and fall off. Finally a round sheet-shaped cell sheet with a diameter of 15-55 mm and a thickness of 10-300 μm is obtained (as shown in FIG. 1), comprising cells with the number of 2×10⁷-8×10⁷, which can be used immediately or cryopreserved for later use.

Compared with single stem cell obtained by traditional trypsin digestion, the umbilical cord mesenchymal stem cell sheet of the present invention is a round sheet-shaped cell junction body, wherein the cells are closely connected to each other and maintain complete ingredients of extracellular matrix (ECM). The ingredients include fibronectin, laminin, collagen, mucopolysaccharides, etc. ECM is an important and basic substance for stem cells to maintain several physiological functions, which contributes to the secretion and signal transmission of cytokines, and is conducive to the colonization and survival of stem cells in the body. Applying the cell sheet to the lesion can 100% transplant umbilical cord mesenchymal stem cells to the lesion, prolong the survival of the stem cells in the body, and exert more effective and lasting therapeutic effects of the stem cells. Umbilical cord mesenchymal stem cell sheets of the present invention are capable of secreting a variety of cytokines after being attached to the lesion, the cytokines mainly including epidermal growth factor (EGF), fibroblast growth factor (FGF), hepatocyte growth factor (HGF), vascular endothelial growth factor (VEGF), transforming growth factor-β (TGF-β), prostaglandin E2 (PGE2), platelet-derived growth factor (PDGF), interleukin 6 (IL-6), interleukin 10 (IL-10) and the like, which can repair the ischemic and damaged tissues and organs or promote their self-repair, thereby achieving the therapeutic effect.

The “chronic heart failure caused by ischemic heart disease and dilated cardiomyopathy” referred to by the present invention includes two categories: one is heart failure caused by myocardial ischemia, including chronic ischemic heart failure and end-stage ischemic heart failure; the other is heart failure caused by dilated cardiomyopathy.

The mechanism of the transplantation of the umbilical cord mesenchymal stem cell sheet in the present invention for the treatment of chronic heart failure caused by ischemic heart disease and dilated cardiomyopathy is: a large number of umbilical cord mesenchymal stem cell sheets secrete a variety of growth factors and cytokines at the transplantation site, mainly including HGF, VEGF, IL-6, IL-10, etc., which regulate the microenvironment of myocardial ischemia site, inhibit the myocardial inflammatory response, prevent the progression of myocardial infarction fibrosis; promote local angiogenesis, provide blood supply to the myocardium through compensatory pathways and improve myocardial function.

The umbilical cord mesenchymal stem cell sheet of the present invention is allogeneic, has excellent performance, and can replace autologous stem cells for the treatment of heart failure. Umbilical cord mesenchymal stem cell sheets show significant advantages: (1) The umbilical cord is widely available, convenient, and non-invasive; (2) Uniform quality standards can be established, best candidates can be selected when taking materials, and the limitations of autologous stem cells due to their own objective conditions can be broken through; (3) Multi-level cell bank and spot products can be prepared in advance to greatly reduce the time for cell culture in vitro and patient waiting; (4) With low immunogenicity of the umbilical cord mesenchymal stem cell sheet, allogeneic application will hardly cause immune rejection; (5) The umbilical cord mesenchymal stem cell sheet has high safety, no tumorigenesis and no toxic reaction; (6) Myocardial ischemia is improved in patients with heart failure through the paracrine effect of stem cells, prolonging the patient survival.

The examples of the present invention are described in detail below, and the illustrations of the examples are shown in the figures. The examples described below with reference to the figures are exemplary, and are intended to explain the present invention, but should not be construed as limiting the present invention.

### Example 1. Preparation of Umbilical Cord Mesenchymal Stem Cell Sheets

The umbilical cord of a human newborn is taken, and the outer membrane and blood vessels are removed to obtain the Wharton's jelly-like tissue within the umbilical cord tissue. The Wharton's jelly-like tissue is cut with a sterile scissor into tissue blocks of about 1-2 mm³, and plated in a petri dish for culture. The tissue blocks are removed after the umbilical cord mesenchymal stem cells migrate out, and fresh medium is added to continue the culture. When the cells grow to about 70-100% confluence, the cells are passaged. Under the microscope, umbilical cord mesenchymal stem cells are observed to grow adherently, appear fibrous and uniform in shape.

The isolated mesenchymal stem cells are identified by detecting the following cell surface markers via flow cytometry: CD105, CD34, CD31 and CD117, where CD105 is a positive marker; CD34, CD31 and CD117 are negative markers. The results show that CD105 is 99.64%, CD34 is 0.02%, CD31 is 0.00%, and CD117 is 0.51% in the isolated umbilical cord mesenchymal stem cells. The results above indicate that the obtained umbilical cord mesenchymal stem cells have high purity.

The ability of umbilical cord mesenchymal stem cells to differentiate into osteocytes and adipocytes is further detected. Specifically, umbilical cord mesenchymal stem cells are seeded in a proportion in a petri dish. For osteogenic induction, osteogenic induction medium is added when the cells grow to about 50-90% confluence and the cells are stained with alizarin red after 7 days of culture; for adipogenic induction, adipogenic induction medium is added when the cells grow to more than 90% confluence, and the cells are stained with oil red 0 after 7 days of culture. As shown in FIG. 2, the mesenchymal stem cells can be stained with alizarin red (FIG. 2A) or oil red 0 (FIG. 2B) after osteogenic induction or adipogenic induction, indicating that they have the ability to differentiate into osteocytes and adipocytes.

For the preparation of the mesenchymal stem cell sheet, the umbilical cord mesenchymal stem cells of P2-P8 passage mentioned above are digested into single cells. After the single-cell suspension is made, it is seeded into a thermo-sensitive petri dish pre-coated with the matrix (such as fetal bovine serum, autologous serum, gelatin, fibronectin, vitronectin, laminin, and polylysine) at the density of 3×10⁵-1.2×10⁶ and cultured in an incubator in an environment of 37° C., 5% CO₂ and 95% humidity. When the cells are cultured to a supersaturated state, they are transferred to an environment of about 20° C. or HBSS solution, PBS solution or saline pre-chilled at 4° C. is added. The lamellar cells are detached from the bottom of the thermo-sensitive petri dish and form a complete cell sheet connected by the ECM. The obtained sheet is round and sheet-shaped, 15-55 mm in diameter, 10-300 μm in thickness, and off-white. They have a dense structure with a smooth and flat surface, the ratio of live cells in the cell sheet is high, and the cells are in good condition (as shown in FIG. 1). They comprise 2×10⁷-8×10⁷ cells and can be used instantly or frozen for future use.

### Example 2. Characterization of Mesenchymal Stem Cell Sheets

The structure of the prepared umbilical cord mesenchymal stem cell sheet is characterized using a scanning electron microscope and immunofluorescence imaging The cell sheet is photographed by scanning electron microscopy after preparation through steps of 2.5% glutaraldehyde fixation, alcohol gradient dehydration and air drying, etc. As shown in FIG. 3, the cell sheet has a surface not contacted with the petri dish (the upper surface, FIG. 3A) and a basal surface contacted with the petri dish (the lower surface, FIG. 3B), with differences in structure: the surface formed is relatively smooth due to the natural sedimentation of cells; the basal surface is contacted with the material of the thermo-sensitive petri dish and is relatively rough. Due to its structural characteristics, the basal surface can provide greater friction, which is beneficial to the cell sheet to be better attached to the application site.

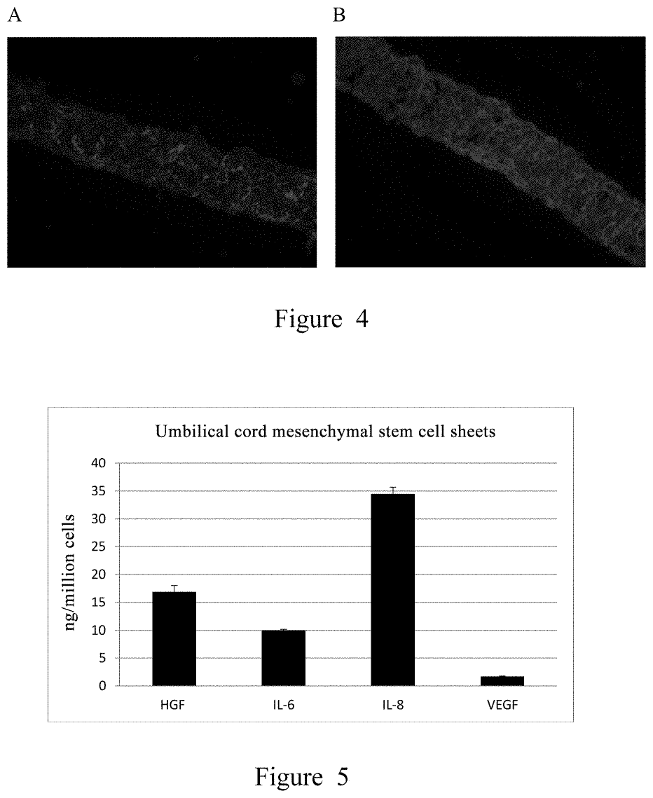

Subsequently, the expressions of fibronectin and integrin-β1 in the umbilical cord mesenchymal stem cell sheet are detected by immunofluorescence. The sheet is fixed by a fixative solution before frozen and sectioned, stained with fluorescein-labeled fibronectin and integrin-β1 antibodies, and analyzed by immunofluorescence imaging. As shown in FIG. 4, the cell sheet prepared by the method of the present disclosure contains a large amount of fibronectin (FIG. 4A) and integrin-β1 (FIG. 4B).

Fibronectin, which is widely present in animal tissues and tissue fluids, has the function of promoting cell adhesion and growth, and cell adhesion and growth are necessary for maintaining and repairing the body tissue structures. Integrin-β1 is an important member of the integrin family, which is important in mediating cell-cell adhesion, cell-cell extracellular matrix (ECM) adhesion and bidirectional signaling, and is closely related to tissue repair and fibrosis formation. The results above indicate that the umbilical cord mesenchymal stem cell sheet disclosed in the present invention is a dense tissue and biologically active sheet formed by the extracellular matrix connections rather than a simple accumulation of cells.

The ability of mesenchymal stem cells to secrete cytokines is further detected, including hepatocyte growth factor (HGF); interleukin-6 (IL-6), interleukin-8 (IL-8) and vascular endothelial growth factor (VEGF). HGF, produced by mesenchymal stem cells, is involved in the epithelial-mesenchymal transition (EMT) process, and has a regulatory effect on a variety of tissues and cells and can promote cell movement and division; IL-6 and IL-8 are involved in regulating immune responses in the body and various physiological processes of immune cells; VEGF has the functions of promoting endothelial cell proliferation and inducing angiogenesis. The cytokines above have the functions of promoting cell growth and differentiation and promoting the process of angiogenesis, and play important roles in tissue repair.

During the preparation of the cell sheet, the culture supernatant is sampled, and the cytokines in the supernatant are detected by the ELISA method. The results are shown in FIG. 5. The results indicate that the above four cytokines are all expressed in the supernatant, and the expression levels of HGF and IL-8 are relatively high. The results above indicate that umbilical cord mesenchymal stem cell sheets of the present disclosure are capable of secreting a variety of cytokines, including angiogenic factors and immunomodulatory factors, demonstrating its high biological activity and function, and can promote local angiogenesis and tissue repair processes. Moreover, the high expression level of IL-8 indicates that the cell sheet has the function of promoting immune response and inhibiting bacteria during use, which is beneficial to the cell sheet to better exhibit its biological function.

### Example 3. Preparation of Fresh Mesenchymal Stem Cell Sheets for Immediate Use

The thermo-sensitive petri dish with mesenchymal stem cell sheet grown in Example 1 is removed from the cell incubator, the medium is aspirated and discarded, and 4° C. pre-chilled PBS or saline is added. After 10 minutes, the cells automatically peel off the edge of the petri dish and gradually expand to the center; if the cells fail to peel off automatically, a 10 μL pipette tip can be used to gently draw a circle along the wall of the petri dish to promote the peeling of the cells. The completely peeled mesenchymal stem cell sheet is transferred to a common petri dish, washed twice with physiological saline, added with 10 mL of fresh sheet protection solution, and aseptically sealed and packaged.

### Example 4. Resuscitation of Cryopreserved Mesenchymal Stem Cell Sheet Spares

The cryopreserved mesenchymal stem cell sheet obtained in Example 1 is removed from the cryogenic container, and quickly placed into a 37° C. incubator or a water bath to quickly thaw within 2 minutes. Thawed mesenchymal stem cell sheet is transferred to a common petri dish, washed twice with physiological saline, added with 10 mL of fresh sheet protection solution, and aseptically sealed and packaged.

### Example 5

### 1) Identification of the Immunogenicity of Mesenchymal Stem Cells

The use of allogeneic umbilical cord mesenchymal stem cells as production materials belongs to allogeneic stem cell transplantation, and immune rejection needs to be considered. To this end, the inventors performed in vitro immunogenicity identification of umbilical cord mesenchymal stem cells.

The inventors detected umbilical cord mesenchymal stem cells by flow cytometry and found that umbilical cord mesenchymal stem cells do not express HLA-II antigens, HLA-DR and co-stimulatory antigens CD80 and CD86; the inventors mixed umbilical cord mesenchymal stem cells with human peripheral blood mononuclear cells at a ratio of 1:2-1:30 and co-cultured them in a complete medium containing 10% fetal bovine serum. After 2 days, the suspended cells in the supernatant were collected for counting and flow cytometry. Abnormal proliferation of lymphocytes was not observed, and no changes in CD3 and CD8 of T cell subsets were observed.

The results of these in vitro experiments indicate that the umbilical cord mesenchymal stem cells are of low immunogenicity.

### 2) Evaluation of Tumor Promotion of Umbilical Cord Mesenchymal Stem Cells

Balb/c nude mice are subcutaneously inoculated with human lung cancer A549 cells to establish a solid tumor model, and NPG nude mice are subcutaneously inoculated with human lymphoma Raji cells to establish a hematoma model. A sufficient dose of umbilical cord mesenchymal stem cells is injected through the tail vein, and the tumor volume is measured for 4 to 8 consecutive weeks. Then, gross anatomy is performed, tumor is weighted, and pathological examination is performed. Compared with the control group, the volume and weight of the tumor in the nude mice in the umbilical cord mesenchymal stem cell injection group are not significantly different. There are no tumors in all major organs of the body, and there are no significant differences in lymphocyte infiltration and the degree of infiltration to surrounding tissues by pathological examination.

This experiment shows that umbilical cord mesenchymal stem cells have no inhibitory or promoting effect on solid tumors and hematomas.

### 3) Evaluation of Tumorigenicity of Mesenchymal Stem Cell Sheet

Balb/c nude mice are subcutaneously transplanted with sufficient doses of the mesenchymal stem cell sheets obtained in Examples 1, 3 or 4 of this application, raised in mouse cages with SPF clean environment, and observed continuously for 20 weeks. No tumor formation is seen at the transplantation site. Gross anatomy is performed to remove the main organs of the nude mice for pathological examination, and no tumor foci are found.

This experiment shows that the mesenchymal stem cell sheet obtained in this application does not have tumorigenicity.

### 4) Evaluation of the Toxicity of Mesenchymal Stem Cell Sheets in Animals

The mesenchymal stem cell sheet of the present invention is different from the traditional administration method of injection of stem cell suspension. It is directly attached to the surface of the heart, will not enter the blood and metastasize, and only plays the role of nourishing myocardium and regulating the microenvironment locally. The scope of influence is under certain control. In order to further clarify its safety, the inventors tested the toxicity of mesenchymal stem cell sheets in mice.

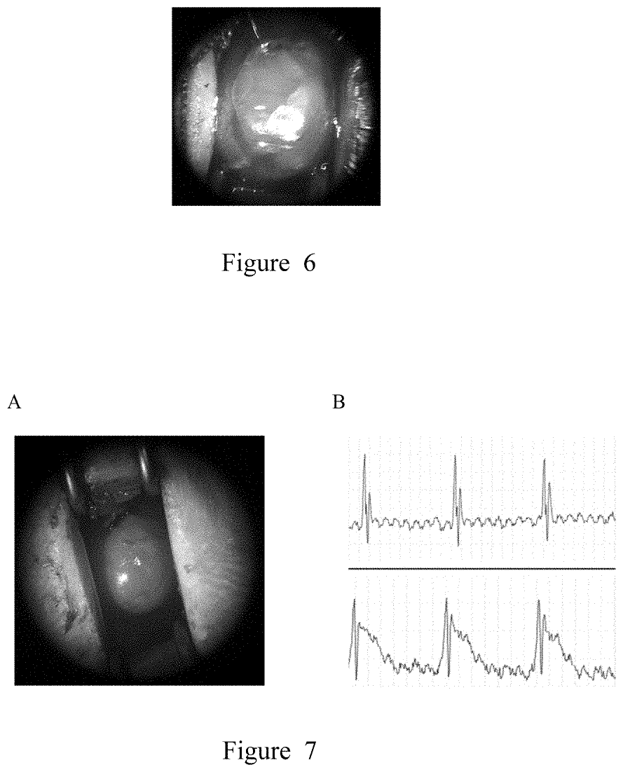

72 NPG mice aged 6-8 weeks were selected with half male and half female, and randomly divided into three groups: cell sheet group, cell suspension group, and negative control group, with 24 mice in each group, 12 females each. The mouse heart in the cell sheet group was transplanted 10 times the dose of mesenchymal stem cell sheets used for human (FIG. 6), the cell suspension group was injected with umbilical cord mesenchymal stem cells via the tail vein at a dose of 1×10⁶ cells per mouse, and the negative control group was injected with an equal volume of 0.9% sodium chloride. Body weight was measured once a week after administration, and observations next to the cage were performed once a day for 4 weeks. The observations included tumor formation at the transplantion site, death, appearance, physical signs, behavioral activity, glandular secretion, respiration, and fecal traits of nude mice. At 24 hours, 1 week, 2 weeks, and 4 weeks after administration, 3 mice of each gender in each group were sacrificed and dissected for gross anatomy observation.

Results: During the experiment, the animals in each group had no abnormalities, survived well, had no difference in body weight, and had no abnormalities in drinking and eating, physical activity, breathing and excretion, and no obvious adverse reactions were seen. Gross anatomy observations were performed at each time point, and no pathological lesions or tumor formation were found.

Conclusion: No obvious acute toxicity is seen upon transplantation of the mesenchymal stem cell sheet obtained in this application into mice.

### Example 6. Construction of Animal Model of Heart Failure

In this example, a mouse model of ischemic heart failure is constructed by coronary artery ligation. In male C57BL/6 mice (approximately 12 weeks old), sutures are used to ligate the left anterior descending branch, which hinder the blood supply of the left ventricle myocardium and lead to myocardial cell apoptosis in the infarct area, resulting in decreased left ventricular ejection function, remodeling of the ventricular structure and eventually heart failure. Specifically, the following steps are included:

(1) The mice are anesthetized with isoflurane mixed with oxygen (isoflurane concentration is about 3.5-5%), and are depilated by using depilatory cream.

(2) Transtracheal intubation is performed through cervical transillumination. The mouse tongue is slightly dragged out using forceps to expose the pharynx. 22G retention needle is inserted into the glottis through the mouth, moves slightly down into the trachea about 5 mm, and exits the retention needle core. Upon blow/inhalation into the retention needle with a Pasteur pipet or a pipette, larger ups and downs can be seen at the thoracic cavity of the mouse, indicating a successful tracheal intubation.

(3) Anesthesia is maintained with a ventilator. The maintenance anesthetic gas is about 3% isoflurane, the tidal volume is 0.3 ml, the frequency is about 124 times/min, and the respiration ratio is 50:50.

(4) ECG signal is measured. A Medlab two-lead physiological signal collection line system is used to monitor the mouse ECG, where the right upper limb is subcutaneously connected to the positive electrode, the left lower limb is subcutaneously connected to the negative electrode, and the right lower limb is subcutaneously grounded.

(5) The chest is opened to expose the heart. The chest skin is opened upward from the xiphoid process using a sharp instrument, the subcutaneous tissue is peeled off, the 4-5th intercostal muscle tissue is pierced with forceps, the ribs are spread with a chest expander, the pericardium is cut open, and the opening range of the chest expander and the position of the mouse are adjusted to fully expose the heart.

(6) The left anterior descending branch is ligated. 6-0 or 7-0 surgical sutures are used to ligate the left anterior descending branch of the mouse, that is, at a distance of about 1.5 mm from the lower edge of the left atrial appendage.

(7) The thoracic cavity is sutured. After the model is completed, the chest expander is removed, the intercostal muscle tissue is returned to its place, and then the epidermis is sutured. The air in the thoracic cavity can be removed by squeezing the thoracic cavity before the suture is completed or sucking the thoracic cavity with a syringe after the suture is completed, so as to avoid the death of the animal caused by pneumothorax.

(8) Postoperative care. Stop the isoflurane input to the ventilator and observe whether the mice breathe spontaneously. If so, move the mouse to a warm blanket to recover until fully awake and able to move on its own. If the mouse is still unable to breathe spontaneously, continue to use the ventilator for assisted breathing until it resumes spontaneous breathing. Only drinking water is provided within 1 hour after surgery, and then feed is provided as usual. When necessary, certain warm measures are given.

After step (6), whitening of the wall of left ventricle of heart in the mouse can be clearly observed (FIG. 7A). The ST segment elevation can be clearly seen on the electrocardiogram, showing an electrocardiogram of myocardial infarction (FIG. 7B). The modeling method can simulate the course of acute ischemic heart failure to a good extent, with high modeling success rate and good stability.

### Example 7. Application of Mesenchymal Stem Cell Sheet in the Treatment of Heart Failure

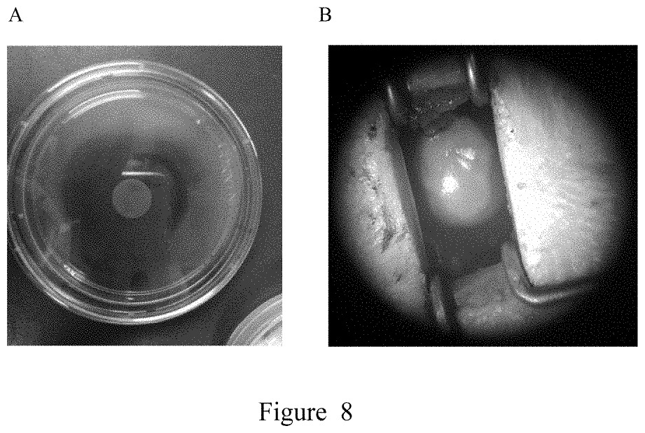

The therapeutic effect of umbilical cord mesenchymal stem cells is evaluated in the disease animal model described in Example 6. For mice in the umbilical cord mesenchymal stem cell sheet treatment group, the umbilical cord mesenchymal stem cell sheet that is cut into a round shape of about 2-5 mm (FIG. 8A) or an appropriate shape with a similar area (FIG. 8B) is attached to the left ventricular surface of the model animal after step (6). The mesenchymal stem cell sheet can be attached well, not easy to fall, and the surface after attachment is flat. The steps (7) thoracic cavity suturing and (8) postoperative care mentioned above are performed after standing for 3-5 minutes Animals with no cell sheet attached are used as controls. 10 mice are in each of the cell sheet treatment group and the control group.

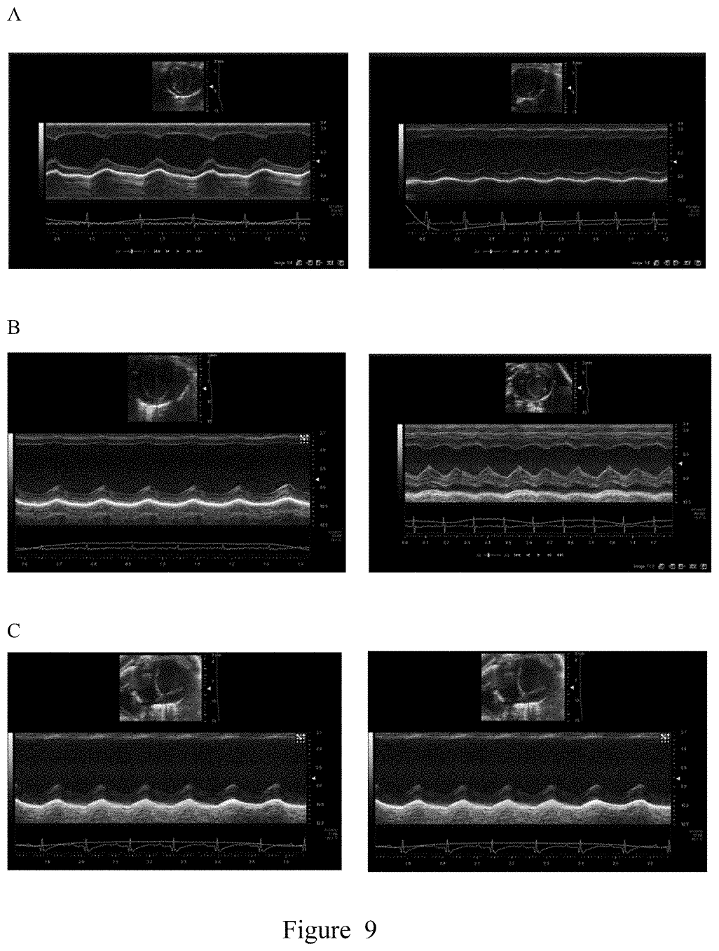

Echocardiography is performed on the mice before modeling (FIG. 9A), 1 week after modeling (FIG. 9B), and 4 weeks after modeling (FIG. 9C). The parasternal short-axis section with the horizontal section of the left ventricular papillary muscle as the mark point, which can be observed by the echocardiography. From the results in FIG. 9B and FIG. 9C, it can be seen that the heart of the heart failure model animal has obvious impaired movement after modeling. In addition, compared to the control group (left panel), the cell sheet treatment group (right panel) has stronger cardiac movement.

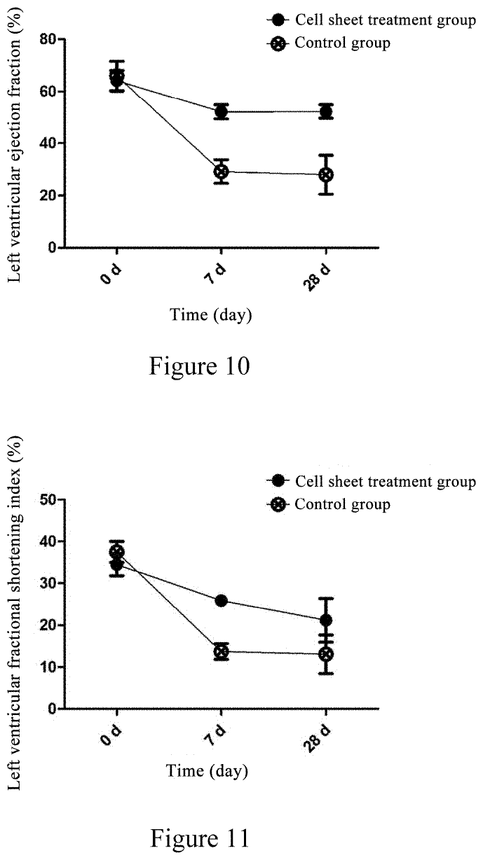

The curves of the left ventricular ejection fraction of mice over time (FIG. 10) and the curves of the left ventricular fractional shortening index of mice over time (FIG. 11) before and after surgery are calculated and plotted according to the echocardiogram. Left ventricular ejection fraction is an important indicator for evaluating left ventricular function. As shown in the results in FIG. 10, the left ventricular ejection fraction of the heart failure model animals significantly decreases after modeling, but animals in the cell sheet treatment group have significantly higher ejection fractions than those in the control group. The left ventricular fractional shortening index refers to the ratio of the short-axes of the left ventricle during systole to diastole. The larger the ratio, the stronger the systolic function of the heart. As shown in the results in FIG. 11, the values of left ventricular fractional shortening index of the heart failure model animals decrease significantly after modeling, but the values of left ventricular fractional shortening index of the animals in the cell sheet treatment group are significantly higher than those in the control group.

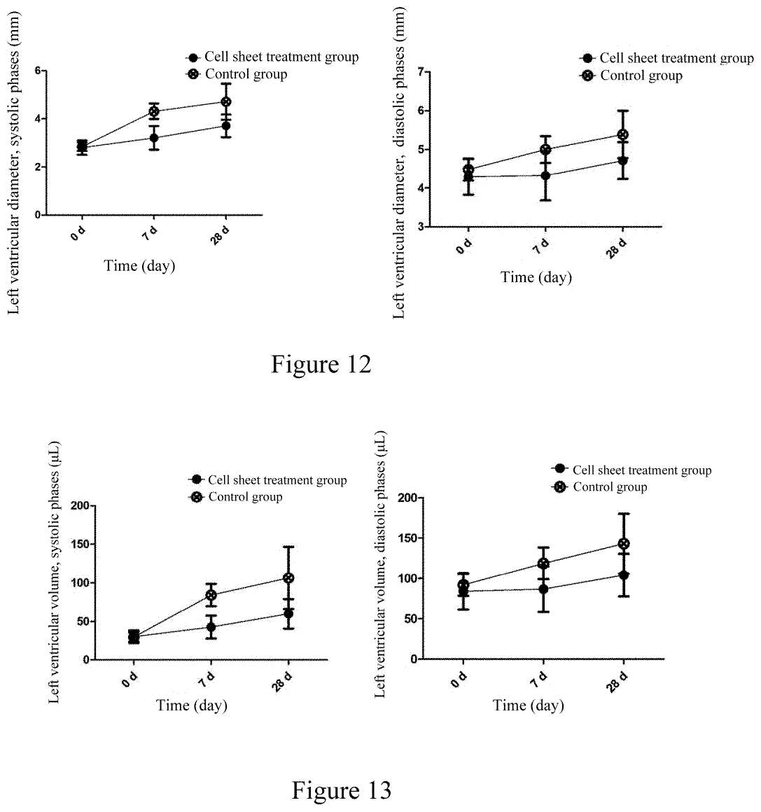

The curves of the left ventricular diameter over time (FIG. 12) and the curves of the left ventricular volume over time (FIG. 13) are calculated and plotted according to the echocardiogram, both of which can be used to describe left ventricular volume. After the animal model is prepared, compensatory remodeling occurs in the left ventricle, and the volume of the ventricle becomes larger due to ischemic heart failure. As shown in the results in FIGS. 12 and 13, after modeling, the left ventricular diameter and volume (systolic and diastolic phases) of the animals in the cell sheet treatment group are significantly lower than those in the control group, indicating that the use of cell sheets has a significant effect on inhibiting left ventricular remodeling caused by ischemic heart failure, and can significantly improve heart function.

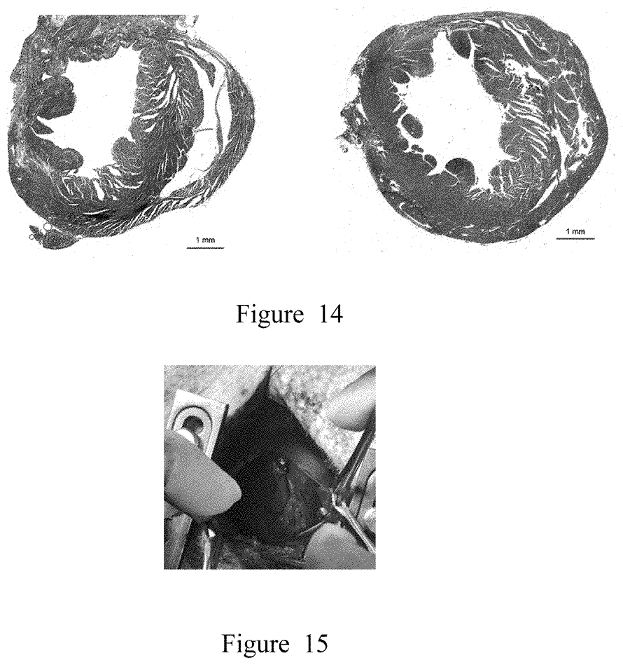

At the end of the experiment (day 28 after modeling), the mice are sacrificed and the heart tissues are taken for fixation, sectioning and staining. The results of sectioning (FIG. 14) show that the left ventricular wall of the mice in the mesenchymal stem cell sheet treatment group is thicker, the ventricular remodeling is less severe, and the degree of fibrosis is lower (Masson staining, collagen fibers appear blue) than the animals in the control group. The results above indicate that the degree of fibrosis in the left ventricle of mice treated with cell sheets is significantly lower than that of control animals.

### Example 8. Mesenchymal Stem Cell Sheet Transplantation on Myocardial Recovery Test of Animal Model

The Bama miniature pig is selected as the experimental animal for establishment of the myocardial infarction model by ligating the anterior descending coronary artery. The modeling method is as follows:

The animal is subjected to induction anesthesia by intramuscular injection of Zoletil, the operation area is skin prepared and the animal is fixed on a constant-temperature animal operating table. The anesthetic animal is subjected to tracheal intubation, connected to an anesthesia ventilator, maintained anesthesia with isoflurane, and changed from spontaneous breathing to passive breathing. The operation area is wiped and disinfected with 4.5 g/L˜5.5 g/L iodophor and 75% alcohol. Tilidine is intramuscularly injected for analgesia.

A high-frequency electrocoagulation knife is used to open between the 4th and 5th ribs on the left side of the chest and separate layer by layer into the thoracic cavity, and the heart is exposed with a thoracic cavity dilator. The left circumflex branch of the left coronary artery is freed, and the coronary artery blocking reperfusion device is implanted and fixed. The outer end of the reperfusion device is blocked, and subcutaneously pulled to the back of the chest or back of the neck and fixed. After that, the pericardium is sutured and the thoracic cavity is closed after injecting an appropriate amount of antibiotics. After the operation, the animals are injected with penicillin sodium intramuscularly.

The next day, when the animal is awake, the animal with the coronary artery blocking reperfusion device implanted is placed in the cloth bag of the fixed frame, and the electrocardiogram of the animal before the coronary artery blocking to post-reperfusion is detected through the jacket-type physiological signal telemetry system.

Determination as successful model of myocardial infarction: the coronary blood flow of the animal is blocked by pressurizing by injecting air or water into the coronary blocking reperfusion device. The myocardial ischemia is confirmed by increase of the ST segment voltage in the electrocardiogram by more than 0.1 mV after blocking of blood flow. One hour after the coronary artery is blocked, the air or water in the coronary artery blocking reperfusion device is drawn out to perform reperfusion of the coronary blood flow. Reperfusion injury is confirmed by continuous elevation of the ST segments in the electrocardiogram, towering of T waves, or the appearance of pathological Q waves.

Two weeks after the formation of the myocardial infarction model, 1-2 mesenchymal stem cell sheets obtained in Examples 1, 3 or 4 of this application are transplanted to the anterior wall of the left ventricle (FIG. 15); the model control group is not transplanted with any materials, with only myocardial infarction modeling and secondary thoracotomy sham operation performed. The observation is continued for 9 weeks after sheet transplantation. Observation indicators include physiological indicators, electrocardiogram, hemodynamics, and echocardiogram. The animals are euthanized for histopathological examination at the end of the experiment. The examination methods are as follows:

The heart is taken out, placed in the sodium chloride injection solution and rinsed gently. 1% TTC solution in 20 mL per animal heated at 37° C. is perfused into the heart through the coronary entrance of the aortic root, and the left circumflex branch is then ligated.

The heart tissue is cut in cross-section, cut into 5-6 pieces of heart tissue with an interval thickness of 5 mm, which are placed in 37° C. TTC solution for incubation for about 5-10 minutes (to allow sufficient TTC reaction, the ischemic tissue is off-white, and the normal tissue is red or bright red). The later slices for the heart tissue are placed facing up and fixed in 10% neutral buffered formalin solution after scanning Photoshop 7.0 is used to select the infarct area for the photographs. NIH Image J software is used to determine the volume of the myocardial infarction area (mm³), the total volume of the left ventricle (mm³) and the infarct rate (%). Volume calculation formula=(front slice area+back slice area)×slice thickness (5 mm)/2, infarct rate=total infarct volume/total left ventricular volume×100%.

The second piece of heart tissue is taken to make a white slice, stained with Masson's three-color method, and subjected to histopathological examination using an optical microscope. The fibrosis degree of myocardial tissue is evaluated. The results of microscopic examination are graded by using 4-grade method, which are slight (+), mild (++), moderate (+++), and severe (++++) for comparison between groups.

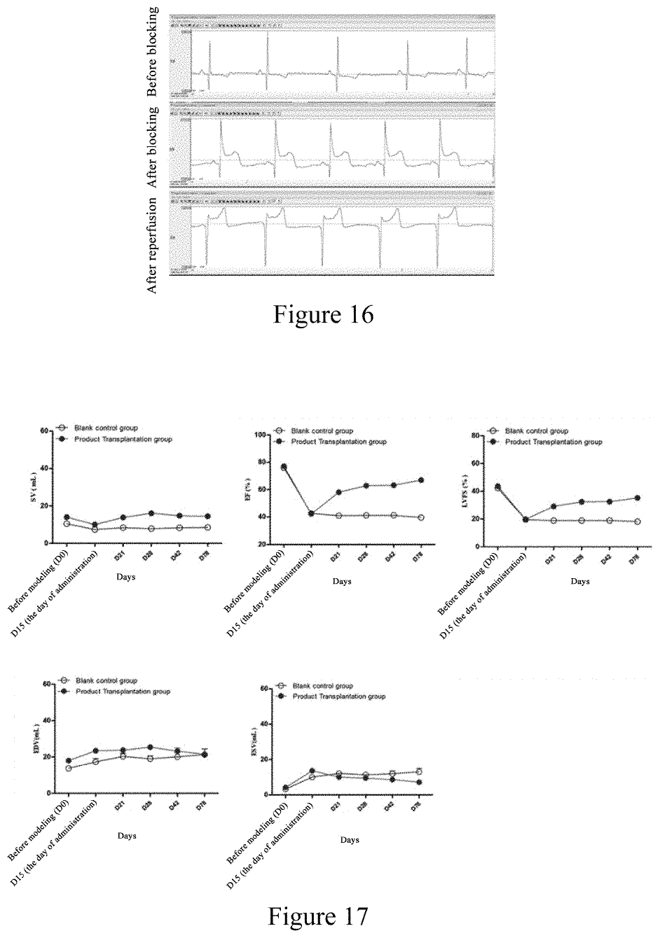

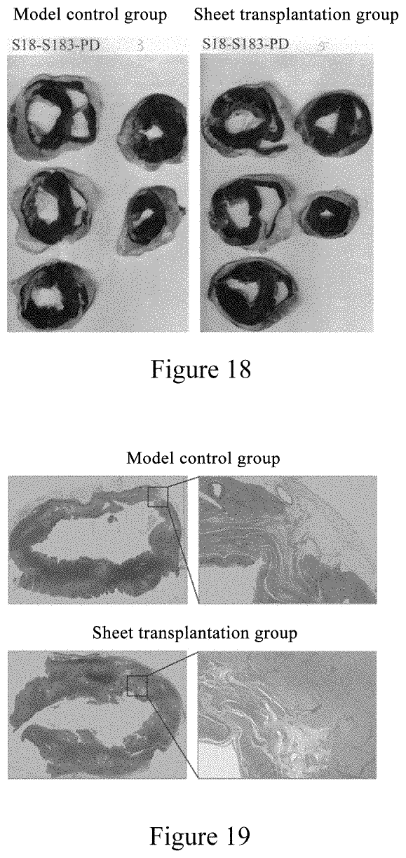

Results: During this experiment, the general clinical observation of animals shows no obvious abnormalities. The weight of the animals in the model control group and the sheet transplantation group increase steadily, and the animals' physiological needs such as food intake and drinking are normal. The electrocardiogram changes are ST segment elevation, T waves towering and pathological Q waves after successful myocardial infarction modeling (FIG. 16). There is no significant difference in left ventricular end systolic pressure (LVESP, mmHg), left ventricular end diastolic pressure (LVEDP, mmHg) and ±dp/dt max indexes between the two groups of experimental animals. The echocardiogram shows that compared with the model control group (blank control group), the EF, SV, LVFS and other indexes of the D21, D28, D42, D78 sheet transplantation group (product transplantation group) animals show a significant increasing trend, with obvious decreasing trend for ESV index and no obvious changes for other indexes (FIG. 17). The pathological examination results show that the myocardial infarction rate of the animals in the sheet transplantation group is significantly reduced (FIG. 18, Table 1), and the degree of myocardial fibrosis is reduced (FIG. 19).

Conclusion: In the myocardial infarction model of miniature pig, transplantation of mesenchymal stem cell sheets can effectively improve heart function and significantly reduce the occurrence of myocardial fibrosis and myocardial infarction volume.

TABLE 1
Comparison of myocardial infarction in miniatue pigs

| | Total | Total left | Infarct |
|---|---|---|---|
| Group | infarct volume | ventricular volume | rate |
| Blank control | 3333.20 ± 1368.67 | 22992.86 ± 4319.41 | 14.2 ± 4.5 |
| group | | | |
| Product | 1381.82 ± 813.77 | 32257.44 ± 4760.11 | 4.0 ± 2.0 |
| transplantation | | | |
| group | | | |

### Example 9. Surgical Method of Open-Thoracic Transplantation of Mesenchymal Stem Cell Sheets

The packages of mesenchymal stem cell sheets from Example 3 or 4 are opened, and the sheet is washed twice with saline at amount of 20 ml for each washing. The patient undergoes a thoracotomy. The pericardium is cut open to expose the left ventricle, and the mesenchymal stem cell sheet is optionally attached to anterior or lateral wall of left ventricle of heart. The dosage for each patient is 1-4 pieces of mesenchymal stem cell sheets, and there may be some overlapping areas during application. Observation is performed for 5-10 minutes after transplantation. After confirming that the mesenchymal stem cell sheet is stable on the surface of the heart, the pericardium can be sutured and the thoracic cavity can be closed.

### Example 10. Method of Transplanting Mesenchymal Stem Cell Sheet in Combination with Coronary Artery Bypass Graft

After the patient undergoes coronary artery bypass graft, the mesenchymal stem cell sheet of Example 1, 3, or 4 is attached to the site undergoing bypass, and the rest of the surgical method is the same as that of Example 9.

### Example 11. Method of Transplanting Mesenchymal Stem Cell Sheets in Minimally Invasive Surgery

The patient undergoes minimally invasive surgery. The thoracic cavity is entered under the guidance of an endoscopy, the pericardium is cut open, the ischemic lesion of anterior wall of left ventricle of heart is accurately found, and the curled mesenchymal stem cell sheet of Example 1, 3, or 4 is released to the lesion site, and is then spread flat. It can be used in multiple pieces with overlapping. The total amount is 1-4 sheets/person.

The specific examples of the present invention are described in detail above, but they are only illustrative, and the present invention is not limited to the specific examples described above. For those skilled in the art, any equivalent modifications and substitutions made to the present invention are also within the scope of the present invention. Therefore, all equivalent changes and modifications made without departing from the spirit and scope of the present invention should fall within the scope of the present invention.

1. A method of treating a disease related to cardiac tissue damage or cardiac insufficiency in a subject, comprising applying an effective amount of a mesenchymal stem cell sheet to the heart of the subject topically.

2. (canceled)

3. The method of claim 1, wherein the disease is selected from ischemic heart disease, rheumatic heart disease, congenital heart disease, cardiomyopathy, coronary heart disease and valvular heart disease.

4. The method of claim 3, wherein the cardiomyopathy is dilated cardiomyopathy.

5. The method of claim 3, wherein the disease is ischemic heart failure.

6. The method of claim 1, wherein the mesenchymal stem cell sheet is applied topically by attaching it to anterior or lateral wall of left ventricle of heart.

7. The method of claim 1, wherein the mesenchymal stem cell sheet is applied topically by attaching or implanting it to damaged or defective sites of heart, or adjacent sites thereof.

8. (canceled)

9. The method of claim 1, wherein the cell ratio of mesenchymal stem cells in the mesenchymal stem cell sheet is at least 90%.

10. The method of claim 1, wherein the mesenchymal stem cells are derived from a tissue selected from: amniotic fluid, amniotic membrane, chorion, chorionic villus, decidua, placenta, umbilical cord blood, Wharton's jelly, umbilical cord, adult bone marrow, dental pulp, adult peripheral blood and adult adipose tissue.

11. The method of claim 1, wherein the mesenchymal stem cells are selected from umbilical cord mesenchymal stem cells, placental mesenchymal stem cells, adipose mesenchymal stem cells, dental pulp mesenchymal stem cells and bone marrow mesenchymal stem cells.

12. The method of claim 11, wherein the mesenchymal stem cells are umbilical cord mesenchymal stem cells.

13. The method of claim 1, wherein the mesenchymal stem cell sheet is prepared by using mesenchymal stem cells with passage number of P2-P10.

14. The method of claim 1, wherein the thickness of the mesenchymal stem cell sheet is 30-300 μm.

15. (canceled)

16. The method of claim 1,
wherein the cell density of the mesenchymal stem cell sheet is 1×10⁵ to 5×10⁷/cm².

17-21. (canceled)

22. The method of claim 1, wherein the mesenchymal stem cell sheet is topically applied to the heart of the subject in combination with coronary artery bypass graft performed on the subject.

23. The use of method of claim 1, wherein the mesenchymal stem cells in the mesenchymal stem cell sheet are autologous or allogeneic to the subject.

24. The method of claim 1, wherein the mesenchymal stem cell sheet is prepared by a method comprising the following steps:
a. culturing the mesenchymal stem cells in a thermo-sensitive petri dish pre-coated with adhesion matrix or serum;
b. detaching the mesenchymal stem cells from the thermo-sensitive petri dish by decreasing the temperature,
wherein the mesenchymal stem cells are connected to each other by extracellular matrix secreted by the cells, thereby obtaining said mesenchymal stem cell sheet.

25. (canceled)

26. The method of claim 24, wherein the adhesion matrix includes one or more of fetal bovine serum, autologous serum, collagen, gelatin, fibronectin, vitronectin, laminin, polyornithine and polylysine.

27. The method of claim 24, wherein the mesenchymal stem cell sheet has a upper surface not contacted with the petri dish and a basal surface contacted with the petri dish during the preparation process, wherein the basal surface is rough, and wherein the basal surface of the mesenchymal stem cell sheet is attached to damaged or defective sites of heart, or adjacent sites thereof, or wherein the basal surface of the mesenchymal stem cell sheet is attached to anterior or lateral wall of left ventricle of heart.

28-29. (canceled)

30. The method of claim 1, wherein the effective amount is a single-dose of one to four mesenchymal stem cell sheets, and wherein 2×10⁷ to 8×10⁷ mesenchymal stem cells are comprised in each of the sheets.

31. The method of claim 30, wherein the diameter of the sheet is 15-55 mm and the thickness of the sheet is 10-300 μm.
